(19) United States
(12) Patent Application Publication
(10) Pub. No.: US 2008/0214611 A1
(43) Pub. Date: Sep. 4, 2008

(54) Pharmaceutical Compositions - 659

(75) Inventors: Lennart LINDFORS, Molndal (SE); Urban Skantze, Molndal (SE)

Correspondence Address: MORGAN LEWIS & BOCKIUS LLP, 1111 PENNSYLVANIA AVENUE NW, WASHINGTON, DC, 20004, US

(73) Assignee: AstraZeneca AB, Sodertalje (SE)

(21) Appl. No.: 12/028,074

(22) Filed: Feb. 8, 2008

Related U.S. Application Data
(60) Provisional application No. 60/888,955, filed on Feb. 9, 2007.

(51) Int. Cl. A61K 31/4545; A61K 31/415; A61K 31/454

(52) U.S. Cl. 514/318; 514/406; 514/326

(57) ABSTRACT

The invention relates to a process for the preparation of a stable dispersion of amorphous particles of a CB1 modulator of sub-micron size in an aqueous medium.

This application claims the benefit under 35 U.S.C. § 119(e) of Application No. 60/888,955 (US) filed on 9 Feb. 2007.

### BRIEF SUMMARY OF THE INVENTION

The present invention relates to a process for the preparation of a stable dispersion of particles, particularly sub-micron particles in an aqueous medium and to a stable dispersion of particles in a liquid medium. More particularly the present invention relates to a process for the preparation of a dispersion of particles comprising an amorphous substantially water-insoluble CB1 antagonist/inverse agonist compound of a high concentration in an aqueous medium, which exhibits reduced crystallisation rate of the substantially water insoluble active compound. Further, the particles exhibit substantially no increase in size upon storage in the aqueous medium, and in particular aqueous dispersions of particles that exhibit substantially no particle growth mediated by Ostwald ripening.

### BACKGROUND OF THE INVENTION

Dispersions of a solid material in a liquid medium are required for a number of different applications including paints, inks, dispersions of pesticides and other agrochemicals, dispersions of biocides and dispersions of pharmacologically active compounds. In the pharmaceutical field many pharmacologically active compounds have very low aqueous solubility, which can result in low bioavailability. The bioavailability of such compounds may be improved by reducing the particle size of the compound, particularly to a sub-micron size, because this improves dissolution rate and hence absorption of the compound. This effect is expected to be even more pronounced using amorphous particles.

The formulation of a pharmacologically active compound as an aqueous suspension, particularly a suspension with a sub-micron particle size, enables the compound to be administered intravenously and thereby provides an alternative route of administration which may increase bioavailability compared to oral administration.

However, there will generally be a differential rate of dissolution if there is a range of particles sizes dispersed in a medium. The differential dissolution rate has an impact on the thermodynamical stability. The smaller particles are thermodynamically unstable relative to the larger particles. This gives rise to a flux of material from the smaller particles to the larger particles. The effect is that the smaller particles dissolve in the medium, whilst material is deposited onto the larger particles thereby giving an increase in particle size. One such mechanism for particle growth is known as Ostwald ripening (Ostwald, Z Phys. Chem. (34), 1900, 495-503). The growth of particles in a dispersion can result in instability of the dispersion during storage due to the sedimentation of particles from the dispersion. It is particularly important that the particle size in a dispersion of a pharmacologically active compound remains constant because a change in particle size is likely to affect the bioavailability and hence the efficacy of the compound. Furthermore, if the dispersion is to be used for intravenous administration, growth of the particles in the dispersion may render the dispersion unsuitable for this purpose. Theoretically particle growth resulting from Ostwald ripening would be eliminated if all the particles in the dispersion were the same size. However, in practice, it is not possible to achieve a completely uniform particle size and even small differences in particle sizes can give rise to particle growth.

Aqueous suspensions of a solid material can be prepared by mechanical fragmentation, for example by milling. U.S. Pat. No. 5,145,684 describes wet milling of a suspension of a sparingly soluble compound in an aqueous medium. However, a major disadvantage using wet milling is contamination from the beads used in the process. Furthermore, mechanical fragmentation is less efficient in terms of particle size reduction when applied to non-crystalline starting material.

U.S. Pat. No. 4,826,689 describes a process for the preparation of uniform sized particles of a solid by infusing an aqueous precipitating liquid into a solution of the solid in an organic liquid under control of temperature and infusion rate, thereby controlling the particle size.

U.S. Pat. No. 4,997,454 describes a similar process in which the precipitating liquid is non-aqueous. However, when the particles have a small but significant solubility in the precipitating medium particle size growth is observed after the particles have been precipitated. To maintain a particular particle size using these processes it is necessary to isolate the particles as soon as they have been precipitated to minimise particle growth. Consequently, particles prepared according to these processes cannot be stored in a liquid medium as a dispersion. Furthermore, for some materials the rate of Ostwald ripening is so rapid that it is not practical to isolate small particles (especially nano-particles) from the suspension.

U.S. Pat. No. 5,100,591 describes a process for preparing particles, comprising a complex between a water insoluble substance and a phospholipid, by co-precipitation of the substance and a phospholipid into an aqueous medium. Generally the molar ratio of phospholipid to substance is 1:1 to ensure that a complex is formed.

U.S. Pat. No. 6,197,349 describes a process for the formation of amorphous particles by melting a crystalline compound and mixing the compound with a stabilising agent, e.g. a phospholipid, and dispersing this mixture in water at elevated temperature using high pressure homogenization, after which the temperature is lowered to e.g. ambient temperature.

WO 03/059319 describes the formation of small particles by adding a solution of a drug dissolved in a water immiscible organic solvent to a template oil-in-water emulsion after which the water immiscible organic solvent is evaporated off. Water is then removed, e.g. using a spray-drying process to obtain a powder.

U.S. Pat. No. 5,700,471 describes a process for producing small amorphous particles in which crystalline material dispersed in water, is heated and subjected to turbulent mixing above the melting temperature, and the resulting melt emulsion is immediately spray-dried or converted into a suspension by cooling. However, such suspensions will exhibit particle growth mediated by Ostwald ripening. Furthermore, according to U.S. Pat. No. 5,700,471 some substances are not amenable to such a process without using an additional organic solvent due to particle agglomeration. One such compound is fenofibrate.

WO 03/013472 describes a precipitation process without the need of water-immiscible solvents for the formation of dispersions of amorphous nanoparticles. The dispersions prepared herein exhibit little or no particle growth mediated by Ostwald ripening after precipitation. The process comprises combining (a) a first solution comprising a substantially water-insoluble substance, a water-miscible organic solvent and an inhibitor with (b) an aqueous phase comprising water thereby precipitating solid particles. The inhibitor is stated to be a non-polymeric hydrophobic organic compound substantially insoluble in water, less soluble in water than the substance, and not being a phospholipid. WO2004/069277 discloses the use of pyrazine CB1 modulators in this precipitation process. WO2004/069226 discloses the use of thiazole CB1 modulators in this precipitation process. WO2004/069227 discloses the use of pyrrole CB1 modulators in this precipitation process.

Co-pending application WO 2007/021228 describes a process for the preparation of a stable dispersion of amorphous particles of sub-micron size in an aqueous medium. The process comprises the following steps:

1) combining
a) an emulsion comprising
a continuous aqueous phase;
an inhibitor;
a stabiliser;
   with
   b) a substantially water-insoluble substance, wherein the ratio of water insoluble substance to inhibitor is below 10:1 (w/w); and
   2) increasing the temperature of the mixture to the vicinity of the melting temperature of the substantially water-insoluble substance.

The mixture may then, during step 2) be kept at this temperature for a time period sufficient for allowing the substantially water insoluble substance to migrate to the oil phase provided by the inhibitor. The inhibitor is suitably completely miscible with the amorphous phase of the substantially water-insoluble substance. The temperature is then lowered, for example, to ambient temperature, and the dispersion of amorphous sub-micron particles is obtained. The dispersion obtained comprises sub-micron particles having a high concentration of the substantially water-insoluble substance. Since the process described is not a precipitation process high concentrations can be obtained in aqueous systems (Vitale et al., Langmuir 19, 4105 (2003)). For substances with melting points above 100° C., the process is performed under pressure, e.g. using a high-pressure reactor, due to the boiling point of the aqueous medium. The particles, i.e. the “sub-micron particles”, obtained by this method have a mean particle size of less than 10 μm, for example less than 5 μm, or less than 1 μm or even less than 500 nm. It is especially preferred that the particles in the dispersion have a mean particle size of from 10 to 500 nm, for example from 50 to 300 nm, or from 100 to 200 nm. The mean size of the particles may be measured using conventional techniques, for example by dynamic light scattering, to obtain the intensity averaged particle size.

It is known that certain CB₁ modulators (known as antagonists or inverse agonists) are useful in the treatment of obesity, psychiatric and neurological disorders (WO01/70700 EP 658,546 and EP 656,354 which includes the compound known as Rimonabant namely, 5-(4-chlorophenyl)-1-(2,4-dichlorophenyl)-4-methyl-N-1-piperidinyl-1H-pyrazole-3-carboxamide). Pyrazoles having anti-inflammatory activity are disclosed in WO 95/15316, WO96/38418, WO97/11704, WO99/64415, EP 418 845 and WO2004050632. 1,5-Diarylpyrazole-3-carboxamide derivatives are disclosed as having CB₁ modulatory activity in U.S. Pat. No. 5,624,941, WO01/29007, WO2004/052864, WO03/020217, US 2004/0119972, Journal of Medicinal Chemistry, 46(4), 642-645 2003, Bioorganic & Medicinal Chemistry Letters, 14(10), 2393-2396 2004, Biochemical Pharmacology, 60(9), 1315-1323 2000, Journal of Medicinal Chemistry, 42(4), 769-776 1999 and U.S. Pat. Appl. Publ. US 2003199536. 1,5-Diarylpyrazole-3-carboxamide derivatives having a fluorinatedalkylsulphonyloxy phenyl substituent are disclosed in WO2005/080343 and WO2006/067428.

WO 03/007887 and WO03/075660 disclose certain 4,5-diarylimidazole-2-carboxamides as CB₁ modulators. WO03/27076 and WO 03/63781 disclose certain 1,2-diarylimidazole-4-carboxamides which are CB₁ modulators. WO03/40107, WO2006/067443 and WO2005/095354 disclose certain 1,2-diarylimidazole-4-carboxamides as being useful in the treatment of obesity and obesity-related disorders.

PCT/GB2006/003695 (WO2007/039740) discloses 4,5,6,7-tetrahydropyrrolo[3,2-c]pyridin-4-one and 4,5-dihydropyrrolo[3,2-c]pyridin-4-one compounds and processes for preparing such compounds, their use as CB1 modulators in the treatment of obesity, psychiatric and neurological disorders, to methods for their therapeutic use and to pharmaceutical compositions containing them.

WO04/48317 discloses the compound Taranabant namely, N-[(1S,2S)-3-(4-chlorophenyl)-2-(3-cyanophenyl)-1-methylpropyl]-2-methyl-2-[[5-(trifluoromethyl)-2-pyridinyl]oxy]-propanamide, as a CB1R inverse agonist.

CB1 modulators as described above tend to have low aqueous solubility and there is a need for a method of increasing the concentration of these compounds per unit volume of medium for in vivo testing and ultimately perhaps for pharmaceutical administration.

### BRIEF DESCRIPTION OF THE FIGURES

FIG. 1 shows the results of an in vivo rat study in terms of the plasma concentration (μM) in the rats as a function of time (h) upon oral administration of a dispersion prepared according to Example 1 (filled circles) and a dispersion comprising crystalline particles prepared by wet milling (open circles).

FIG. 2 shows the results of an in vivo rat study in terms of the plasma concentration (μM) in the rats as a function of time (h) upon oral administration of a dispersion prepared according to Example 2 (filled circles) and a dispersion comprising crystalline particles prepared by wet milling (open circles).

### DETAILED DESCRIPTION OF THE INVENTION

We have surprisingly found that stable dispersions of amorphous sub-micron particles may be prepared by a process where a substantially water-insoluble CB1 modulator is mixed with a continuous aqueous phase comprising a component inhibiting growth of particles dispersed in an aqueous medium due to flux of material between the particles, in particular particle growth according to the above-disclosed Ostwald ripening mechanism. This component is herein referred to as “the inhibitor”. The mixture obtained is treated for allowing the substantially water insoluble CB1 modulator to migrate into the oily phase formed by the inhibitor. Thus the process according to the invention is without precipitation which is advantageous when working in larger scales. According to one aspect of the present invention there is provided a process for the preparation of a stable dispersion of amorphous particles of a CB1 modulator of sub-micron size in an aqueous medium. The process comprises the following steps:

1) combining
a) an emulsion comprising
an aqueous medium providing a continuous aqueous phase;
an inhibitor providing an oil phase and inhibiting particle growth due to flux of material between the particles dispersed in the aqueous medium;
a stabiliser preventing aggregation of emulsion droplets and optionally particles; with
   b) a substantially water-insoluble CB1 modulator in amorphous and/or crystalline state, wherein the ratio of water insoluble substance to inhibitor is below 10:1 (w/w); and
   c) optionally a second stabiliser preventing aggregation of emulsion droplets and/or said particles,
   2) if any CB1 modulator in the crystalline state is present, increasing the temperature of the resulting mixture to the vicinity of the melting temperature of the crystalline CB1 modulator, and
   3) allowing the CB1 modulator to migrate to said oil phase, and if the temperature was increased in step 2), decreasing the temperature, e.g. to ambient temperature, thereby providing said dispersion of amorphous particles. The mixture may during step 2) be kept at this temperature for a time period sufficient for allowing the substantially water insoluble CB1 modulator to migrate to the oil phase provided by the inhibitor.

In embodiments wherein the CB1 modulator is added in its amorphous state, the temperature may be increased above ambient temperature, i.e. about 20 to 25° C., in order to speed up the migration of CB1 modulator to the emulsion droplets.

For substances with melting points above 100° C., the process is performed under pressure, e.g. using a high-pressure reactor, due to the boiling point of the aqueous medium.

The particles, i.e. the “sub-micron particles”, obtained by the method of the invention have a mean particle size of less than 10 μm, for example less than 5 μm, or less than 1 μm or even less than 500 nm. It is especially preferred that the particles in the dispersion have a mean particle size of from 10 to 500 nm, for example from 50 to 300 mn, or from 100 to 200 nm. The mean size of the particles may be measured using conventional techniques, for example by dynamic light scattering, to obtain the intensity averaged particle size.

Amorphous particles will eventually revert to a thermodynamically more stable crystalline form upon storage as an aqueous dispersion. The time required for such particles to crystallise is dependent upon the components of the particles and the dispersion of the pharmaceutically active compound and may vary from a few hours to a number of weeks. Generally such re-crystallisation will also result in particle growth. The formation of larger crystalline particles is unsuitable for pharmaceutical administration and they are also prone to sedimention from the dispersion. The conversion of the amorphous substance to crystalline substance by crystal nucleation and growth is generally difficult to control. However, according to the present invention, completely miscible amorphous drug/inhibitor systems (including inhibitor mixtures comprising at least one inhibitor and at least one co-inhibitor), enables not only a possibility to influence crystal nucleation but also a reduced crystal growth rate. These advantages are obtained by having a ratio of water-insoluble substance to inhibitor below 10:1 (w/w), for example 4:1, or 2:1 (w/w).

The sub-micron dispersion obtained by the process of the invention is stable, by which we mean that the particles in the dispersion exhibit reduced or substantially no particle growth mediated by flux of material from the smaller particles to the larger particles, for instance explained by the Ostwald ripening mechanism, as well as that the amorphous CB1 modulator exhibits reduced or substantially no crystallization upon storage thereof. The sub-micron dispersion is thus stable in the meaning of remaining in the amorphous state during a considerable long time, i.e. the crystallization rate is reduced significantly.

By the term “reduced crystallisation” is meant that the rate of crystallization in the obtained dispersion of amorphous particles is reduced compared to particles prepared using a similar process but without the use of an inhibitor. Moreover, the rate of crystallisation of said particles is reduced by the use of a higher inhibitor/drug ratio compared to particles prepared using a lower inhibitor/drug ratio.

By the term “reduced particle growth” is meant that the rate of particle growth mediated by flux of material between particles, such as in accordance with the Ostwald ripening mechanism, is reduced compared to particles prepared using a similar process but without the use of an inhibitor. By the term “substantially no particle growth” is meant that the mean size of the particles in the aqueous medium does not increase by more than 10%, for example not more than 5%, over a period of 1 hour at ambient temperature after the formation according to the present process. Preferably the particles exhibit substantially no particle growth.

The presence of the inhibitor together with the substantially water-insoluble CB1 modulator significantly reduces or eliminates particle growth mediated by Ostwald ripening, as hereinbefore described.

When the emulsion and the substantially water-insoluble CB1 modulator is mixed and the temperature is increased as described in step 2) of the process, the substantially water-insoluble CB1 modulator is transported to the phase comprising the inhibitor. It is therefore believed that the inhibitor system should be completely miscible, with the amorphous phase of the substantially water-insoluble CB1 modulator.

To achieve the improved stability of the amorphous submicron particles preferably all crystalline CB1 modulator, if present, is transferred to the amorphous state. This is performed by increasing the temperature in step 2) to the vicinity of the melting temperature of the substantially water-insoluble CB1 modulator, for example suitably to a temperature of ±20° C. of its melting point, or ±15° C. of its melting point, or ±10° C. of its melting point, or ±5° C. of its melting point, allowing the CB1 modulator to migrate to the oil phase and decreasing the temperature below said vicinity of the melting temperature. In case that not all of the crystalline material is transferred into amorphous state the remaining crystalline material may act as seeds for crystallisation.

The process according to the present invention enables stable dispersions of very small particles, especially submicron particles, to be prepared at high concentration without the need to quickly isolate the particles form the liquid medium to prevent particle growth. With “high concentration” is here meant above 1% by weight, such as between 1 to 30% by weight, of the total concentration of the substantially water-insoluble CB1 modulator in the dispersion of the invention, for example 5, 10, 15, 20 or 25% by weight. As said before, the amorphous particles may exhibit crystallisation i.e. the amorphous substance in the particles formed may be transferred from amorphous state to crystalline state, a process which is due to thermodynamic rules. However, the rate of this thermodynamically determined process may be lowered by decreasing the ratio of water-insoluble CB1 modulator to inhibitor being below 10:1 (w/w), for example 9:1, 8:1, 7:1, 6:1, 5:1, 4:1, 3:1, 2:1, or 1:1 (w/w). By decreasing this ratio, the bulk concentration, i.e. the amorphous solubility, in the dispersion of amorphous submicron particles can be lowered. The amorphous solubility in, for example, water may be determined by measuring static light scattering as a function of dilution of the amorphous suspension of the water-insoluble CB1 modulator by adding small volumes of the amorphous dispersion of water-insoluble CB1 modulator successively to a fluorescence cuvette containing water to give the desired concentrations. The optimal ratio is depending upon the water-insoluble CB1 modulator and the inhibitor or inhibitor/co-inhibitor selected.

The invention also provides a process where particles of the same size are obtained even when the concentration of the water-insoluble CB1 modulator varies between the particles. Particles obtained in the process according to the present invention are independent of nucleation, and differ from particles obtained by precipitation type processes.

### The CB1 Modulator

In one embodiment of the invention, the emulsion is mixed with the particles of substantially water-insoluble CB1 modulator which being initially in crystalline state, including one or more crystal forms.

In one embodiment, the water insoluble CB1 modulator is added to the emulsion in an amorphous form. The water-insoluble CB1 modulator in amorphous form may be obtained, for example, by spray-drying, spray-freezing, freeze-drying or spray-granulation. This list of methods for drying is non-exhaustive. Furthermore, the process of the invention is also suitable for amorphous CB1 modulator not available in crystalline state.

In still another embodiment, the water-insoluble CB1 modulator is added to the emulsion as a mixture of CB1 modulator in crystalline state and CB1 modulator in amorphous state.

Thus, the CB1 modulator added to the emulsion is in a state selected from the group consisting of crystalline state, amorphous state, and any mixture thereof.

These crystalline and/or amorphous particles may be of any size of 1 μm or above, for example between 1 μm and 500 μm or between 1 μm and 200 μm.

In one embodiment the (crystalline and/or amorphous) particles of water-insoluble CB1 modulator are first prepared as a suspension in an aqueous phase, optionally containing one or more stabilisers (herein referred to as second stabiliser), optionally the stabiliser may also be in combination with other water-miscible solvents. The aqueous phase may consist of water, or of water in mixture of one or more water miscible organic solvents.

As will be understood, the selection of water-miscible organic solvent will be dependent upon the nature of the substantially water-insoluble CB1 modulator. Examples of such water-miscible solvents include water-miscible alcohol, for example methanol, ethanol, n-propyl alcohol, isopropyl alcohol, tert-butyl alcohol, ethylene glycol; dimethylsulfoxide, a water-miscible ether, for example tetrahydrofuran, a water-miscible nitrile, for example, acetonitrile; a water-miscible ketone, for example acetone or methyl ethyl ketone; an amide, for example dimethylacetamide, dimethylformamide, or a mixture of two or more of the above mentioned water-miscible organic solvents. Preferred water-miscible organic solvents are ethanol, dimethylsulfoxide, dimethylacetamide.

By “substantially water insoluble” is meant a CB1 modulator that has a solubility in water at 25° C. of less than 0.5 mg/ml, preferably less than 0.1mg/ml and especially less than 0.05 mg/ml.

In a preferred embodiment the CB1 modulator has a solubility in the range of from 0.005 μg/ml to 0.5 mg/ml, for example from 0.05 μg/ml to 0.05 mg/ml. The greatest effect on inhibition of particle growth due to flux of material, such as Ostwald ripening inhibition, is observed when the CB1 modulator has a solubility in water at 25° C. of more than 0.05 μg/ml.

The solubility of the CB1 modulator in the crystalline state in water may be measured using a conventional technique. For example, a saturated solution of the CB1 modulator is prepared by adding an excess amount of the substance to water at 25° C. and allowing the solution to equilibrate for 48 hours. Excess solids are removed by centrifugation or filtration and the concentration of the CB1 modulator in water is determined by a suitable analytical technique such as HPLC.

By the invention, a process for producing sub-micron particles comprising a substantially water-insoluble CB1 modulator having a melting point of up to 300° C. is provided. For example the substantially water insoluble CB1 modulator has a melting point below 250° C., such as below 200° C., or below 175° C., such as 150° C.

In one aspect the CB1 modulator is a CB1 antagonist or inverse agonist as described in the patent and literature references listed earlier, including Rimonabant and Taranabant.

In a further aspect the CB1 modulator is a compound of formula (I)

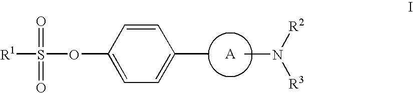

in which
R¹ represents a C₃₋₆alkyl group optionally substituted by one or more fluoro;
R² represents H and R³ represents cyclohexyl optionally substituted by hydroxy or R² and
R³ together with the nitrogen atom to which they are attached represent a piperidine ring which is optionally substituted by hydroxy;

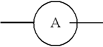

represents a group of formula a, b or c

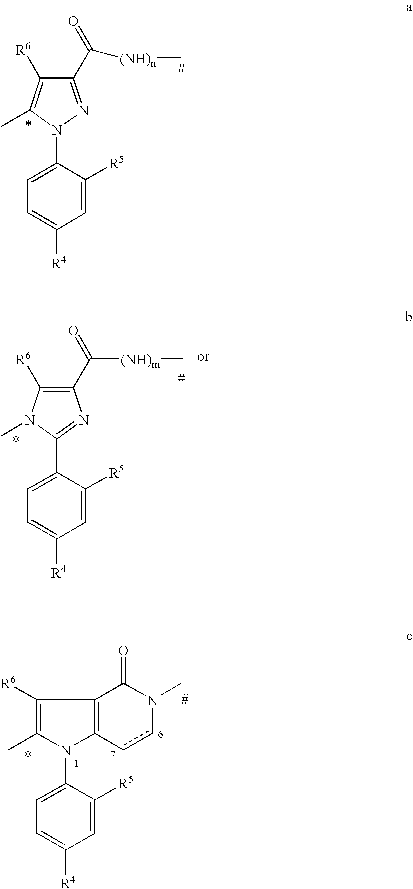

in which the bond marked * is attached to the phenyl ring carrying the sulphonyloxy group and the other bond marked # is attached to NR²R³; [symbol]
is an optional additional bond between positions 6 and 7 in formula c;
R⁴ and R⁵ independently represent H, bromo, chloro or fluoro; and
R⁶ represents methyl or hydroxymethyl;
n and m independently represent 0 or 1;
or a pharmaceutically acceptable salt thereof.

In another aspect the CB1 modulator is a compound selected from:
1-propanesulfonic acid, 3,3,3-trifluoro-, 4-[1-(2,4-dichlorophenyl)-3-[[(2-hydroxycyclohexyl)amino]carbonyl]-4-(hydroxymethyl)-1H-pyrazol-5-yl]phenyl ester;
1-propanesulfonic acid, 3,3,3-trifluoro-, 4-[3-[(cyclohexylamino)carbonyl]-1-(2,4-dichlorophenyl)-4-(hydroxymethyl)-1H-pyrazol-5-yl]phenyl ester;
1-propanesulfonic acid, 3,3,3-trifluoro-, 4-[1-(2,4-dichlorophenyl)-4,5,6,7-tetrahydro-3-methyl-4-oxo-5-(1-piperidinyl)-1H-pyrrolo[3,2-c]pyridin-2-yl]phenyl ester;
1-propanesulfonic acid, 3,3,3-trifluoro-, 4-[1-(2,4-dichlorophenyl)-4-methyl-3-[(1-piperidinylamino)carbonyl]-1H-pyrazol-5-yl]phenyl ester;
1-propanesulfonic acid, 4-[1-(2,4-dichlorophenyl)-4-methyl-3-[(1-piperidinylamino)carbonyl]-1H-pyrazol-5-yl]phenyl ester;
3,3,3-trifluoropropane-1-sulfonic acid, 4-[2-(2,4-dichlorophenyl)-5-methyl-4-(piperidin-1-ylcarbamoyl)imidazol-1-yl]phenyl ester; or
3,3,3-trifluoropropane-1-sulfonic acid 4-[1-(2-chloro-4-fluorophenyl)-3-methyl-4-oxo-5-piperidin-1-yl-4,5,6,7-tetrahydro-1H-pyrrolo[3,2-c]pyridin-2-yl]phenyl ester or a pharmaceutically acceptable salt thereof.

### The Emulsion

The emulsion of the present invention is an emulsion comprising a continuous aqueous phase and an oil phase provided by the inhibitor, i.e. when water is chosen as the continuous aqueous phase, an oil-in-water emulsion. When water, or water in admixture with a water-miscible solvent, is used in the process according to the invention, an emulsion comprising the inhibitor is formed. The emulsion is an oil-in-water emulsion.

The emulsion may also comprise further components as defined below. The emulsion is produced by conventional methods, for example, the inhibitor, a stabilizer and water forms a mixture before it is then homogenised. The homogenisation is performed, for instance, by sonication or high-pressure homogenisation. Preferably, the process of the invention is an aqueous based process wherein the aqueous medium of the continuous aqueous phase consists of water. However, other options for the continuous aqueous phase are also possible, for example, water mixed with a water-miscible solvent. The water miscible solvent may be chosen from the list above or mixture thereof. Further, other options for the aqueous phase may be mixtures of water and low molecular-weight sugars. Such components are added in order to promote the conversion of the amorphous suspension to the dry state e.g. by lyophilisation, spray-drying or spray-granulation. The use of water is an important aspect from an environmental perspective. A water-based process is also advantageous as traces of organic solvent in the particles can be avoided.

### The Stabiliser

The emulsion also comprises at least one stabiliser which prevents aggregation of the emulsion droplets. In a similar way the amorphous particles tend to aggregate in the final dispersion unless a stabiliser is present. Thus, the stabiliser(s) preventing aggregation of the emulsion droplets may suitably also prevent aggregation of the amorphous particles in the resulting dispersion. An alternative is that the emulsion comprises at least one stabiliser preventing aggregation of emulsion droplets and at least one stabiliser preventing aggregation of said particles. Another alternative is that at least one second stabiliser preventing aggregation of said particles is added to the mixture of said emulsion and the CB1 modulator. Still another alternative is that said at least one second stabiliser is added together with the CB1 modulator in a suspension thereof.

Stabilisers suitable for the prevention of emulsion droplet and/or particle aggregation in dispersions are well known to those skilled in the art. Suitable stabilisers include dispersants and surfactants (which may be anionic, cationic or non-ionic) or a combination thereof. Suitable dispersants include, a polymeric dispersant, for example a polyvinylpyrrolidone, a polyvinylalcohol or a cellulose derivative, for example hydroxypropylmethyl cellulose, hydroxy ethyl cellulose, ethylhydroxyethyl cellulose or carboxymethyl cellulose. Suitable anionic surfactants include alkyl and aryl sulphonates, sulphates or carboxylates, such as an alkali metal alkyl and aryl sulphonate or sulphate, for example, sodium dodecyl sulphate or docusate sodium. Suitable cationic surfactants include quaternary ammonium compounds and fatty amines. Suitable non-ionic surfactants include, monoesters of sorbitan which may or may not contain a polyoxyethylene residue, ethers formed between fatty alcohols and polyoxyethylene glycols, polyoxyethylene-polypropylene glycols, an ethoxylated castor oil (for example Cremophor EL), ethoxylated hydrogenated castor oil, ethoxylated 12OH-stearic acid (for example Solutol HS15), phospholipids, for example phospholipids substituted by chains of polyethylene glycols(PEG). Examples are DPPE-PEG (dipalmitoyl phosphatidylethanolamine substituted with PEG2000 or PEG5000 or DSPE-PEG5000 (distearoyl phosphatidylethanolamine substituted by PEG5000). The stabiliser present in the aqueous phase may be a single stabiliser or a mixture of two or more stabilisers. In a preferred embodiment the aqueous phase contains a polymeric dispersant and a surfactant (preferably an anionic surfactant), for example a polyvinylpyrrolidone and sodium dodecyl sulphate or a polyvinylpyrrolidone and docusate sodium. It is preferred that the stabiliser is a pharmaceutically acceptable material.

Generally the aqueous phase will contain from 0.01 to 10% by weight, for example 0.01 to 5% by weight, preferably from 0.05 to 3% by weight and especially from 0.1 to 2% by weight of stabiliser.

### The Inhibitor

The emulsion comprises at least one inhibitor providing an oil phase and inhibiting particle growth due to flux of material between the amorphous particles in the dispersion obtained by the process of the invention.

Suitably for the present invention, the inhibitor fulfils the following:

the inhibitor is a compound that is substantially insoluble in water;
the inhibitor is less soluble in water than the substantially water-insoluble CB1 modulator; and
the inhibitor is completely miscible with the amorphous phase of the substantially water-insoluble CB1 modulator.

It is of importance for the present invention that the inhibitor(s), or the hereinafter described inhibitor mixture (comprising at least one inhibitor and at least one co-inhibitor), affecting particle growth, such as Ostwald ripening, is completely miscible with the amorphous drug. As in WO 03/013472, the miscibility may be characterised by the Bragg-Williams interaction parameter χ. A value of χ being less than 2.5, more preferable χ less than 2 can characterize full miscibility between an amorphous drug and a particle growth inhibitor, i.e. an Ostwald ripening inhibitor.

The inhibitor is suitably a compound that is less soluble in water than the substantially water-insoluble CB1 modulator. Preferably, the inhibitor is a hydrophobic organic compound. The inhibitors suitable for the process of the invention have an influence of the particle growth mediated by Ostwald ripening, as described in WO 03/013472.

Suitable inhibitors have water solubility at 25° C. of less than 0.1 mg/l, more preferably less than 0.01 mg/l. In an embodiment of the invention the solubility of the inhibitor in water at 25° C. is less than 0.05 g/ml, for example from 0.1 ng/ml to 0.05 μg/ml.

In an embodiment of the invention the inhibitor has a molecular weight of less than 2000, for example less than 1000. In another embodiment of the invention the inhibitor has a molecular weight of less than 1000, for example less than 600. For example, the inhibitor may have a molecular weight in the range of from 200 to 2000, preferably a molecular weight in the range of from 400 to 1000, more preferably from 400 to 600.

Suitable inhibitors include an inhibitor selected from classes (i) to (vi) described below, or a combination of two or more such inhibitors:

(i) a mono-, di- or (more preferably) a tri-glyceride of a fatty acid. Suitable fatty acids include medium chain fatty acids containing from 8 to 12, more preferably from 8 to 10 carbon atoms or long chain fatty acids containing more than 12 carbon atoms, for example from 14 to 20 carbon atoms, more preferably from 14 to 18 carbon atoms. The fatty acid may be saturated, unsaturated or a mixture of saturated and unsaturated acids. The fatty acid may optionally contain one or more hydroxyl groups, for example ricinoleic acid. The glyceride may be prepared by well known techniques, for example, esterifying glycerol with one or more long or medium chain fatty acids. In a preferred embodiment the inhibitor is a mixture of triglycerides obtainable by esterifying glycerol with a mixture of long or, preferably, medium chain fatty acids. Mixtures of fatty acids may be obtained by extraction from natural products, for example from a natural oil such as palm oil. Fatty acids extracted from palm oil contain approximately 50 to 80% by weight decanoic acid and from 20 to 50% by weight of octanoic acid. The use of a mixture of fatty acids to esterify glycerol gives a mixture of glycerides containing a mixture of different acyl chain lengths. Long and medium chain triglycerides are commercially available. For example, a preferred medium chain triglyceride (MCT) containing acyl groups with 8 to 12, more preferably 8 to 10 carbon atoms is prepared by esterification of glycerol with fatty acids extracted from palm oil, giving a mixture of triglycerides containing acyl groups with 8 to 12, more preferably 8 to 10 carbon atoms. This MCT is commercially available as Miglyol 812N (Sasol, Germany). Other commercially available MCT's include Miglyol 810 and Miglyol 818 (Sasol, Germany). A further suitable medium chain triglyceride is trilaurine (glycerol trilaurate). Commercially available long chain trigylcerides include soya bean oil, sesame oil, sunflower oil, castor oil or rape-seed oil.

Mono and di-glycerides may be obtained by partial esterification of glycerol with a suitable fatty acid, or mixture of fatty acids. If necessary the mono- and di-glycerides may be separated and purified using conventional techniques, for example by extraction from a reaction mixture following esterification. When a mono-glyceride is used it is preferably a long-chain mono glyceride, for example a mono glyceride formed by esterification of glycerol with a fatty acid containing 18 carbon atoms;

(ii) a fatty acid mono- or (preferably) di-ester of a C₂₋₁₀ diol. Preferably the diol is an aliphatic diol which may be saturated or unsaturated, for example a C₂₋₁₀-alkane diol which may be a straight chain or branched chain diol. More preferably the diol is a C₂₋₆-alkane diol which may be a straight chain or branched chain, for example ethylene glycol or propylene glycol. Suitable fatty acids include medium and long chain fatty acids described above in relation to the glycerides. Preferred esters are di-esters of propylene glycol with one or more fatty acids containing from 8 to 10 carbon atoms, for example Miglyol 840 (Sasol, Germany);
(iii) a fatty acid ester of an alkanol or a cycloalkanol. Suitable alkanols include C₁₋₁₀-alkanols, more preferably C₂₋₆-alkanols which may be straight chain or branched chain, for example ethanol, propanol, isopropanol, n-butanol, sec-butanol or tert-butanol. Suitable cycloalkanols include C₃₋₆-cycloalkanols, for example cyclohexanol. Suitable fatty acids include medium and long chain fatty acids described above in relation to the glycerides. Preferred esters are esters of a C₂₋₆-alkanol with one or more fatty acids containing from 8 to 10 carbon atoms, or more preferably 12 to 29 carbon atoms, which fatty acid may be saturated or unsaturated. Suitable esters include, for example isopropyl myristate or ethyl oleate;
(iv) a wax. Suitable waxes include esters of a long chain fatty acid with an alcohol containing at least 12 carbon atoms. The alcohol may be an aliphatic alcohol, an aromatic alcohol, an alcohol containing aliphatic and aromatic groups or a mixture of two or more such alcohols. When the alcohol is an aliphatic alcohol, it may be saturated or unsaturated. The aliphatic alcohol may be straight chain, branched chain or cyclic. Suitable aliphatic alcohols include those containing more than 12 carbon atoms, preferably more than 14 carbon atoms especially more than 18 carbon atoms, for example from 12 to 40, more preferably 14 to 36 and especially from 18 to 34 carbon atoms. Suitable long chain fatty acids include those described above in relation to the glycerides, preferably those containing more than 14 carbon atoms especially more than 18 carbon atoms, for example from 14 to 40, more preferably 14 to 36 and especially from 18 to 34 carbon atoms. The wax may be a natural wax, for example bees wax, a wax derived from plant material, or a synthetic wax prepared by esterification of a fatty acid and a long chain alcohol. Other suitable waxes include petroleum waxes such as a paraffin wax;
(v) a long chain aliphatic alcohol. Suitable alcohols include those with 6 or more carbon atoms, more preferably 8 or more carbon atoms, such as 12 or more carbon atoms, for example from 12 to 30, for example from 14 to 20 carbon atoms. It is especially preferred that the long chain aliphatic alcohol has from 6 to 20, more especially from 6 to 14 carbon atoms, for example from 8 to 12 carbon atoms. The alcohol may be straight chain, branched chain, saturated or unsaturated. Examples of suitable long chain alcohols include, 1-hexanol, 1-decanol, 1-hexadecanol, 1-octadecanol, or 1-heptadecanol (more preferably 1-decanol); or
(vi) a hydrogenated vegetable oil, for example hydrogenated castor oil.

In one embodiment of the present invention the inhibitor is selected from a medium chain triglyceride and a long chain aliphatic alcohol containing from 6 to 12, preferably from 10 to 20 carbon atoms. Preferred medium chain triglycerides and long chain aliphatic alcohols are as defined above. In a preferred embodiment the inhibitor is selected from a medium chain triglyceride containing acyl groups with from 8 to 12 carbon atoms or a mixture of such triglycerides (preferably Miglyol 812N) and an aliphatic alcohol containing from 10 to 14 carbon atoms (preferably 1-decanol) or a mixture thereof (for example a mixture comprising Miglyol 812N and 1-decanol).

Suitably, the inhibitor is liquid at ambient temperature (25° C.). The inhibitor is preferably a pharmaceutically inert material. The quantity of inhibitor in the particles is sufficient to prevent Ostwald ripening of the particles in the suspension. Preferably the inhibitor will be the minor component in the amorphous particles formed in the present process comprising the inhibitor and the substantially water-insoluble CB1 modulator. Preferably, therefore, the inhibitor is present in a quantity that is just sufficient to prevent Ostwald ripening and to reduce the crystallisation rate to an acceptable level.

Suitable, the inhibitor is compatible with the substantially water-insoluble CB1 modulator, i.e the water-insoluble CB1 modulator in its amorphous phase is miscible with the inhibitor. One way to define miscibility of a water-insoluble CB1 modulator and an inhibitor in the solid particles obtained by the present process is by the interaction parameter χ for the mixture of CB1 modulator and inhibitor. Generally, the amorphous state of the substantially water-insoluble CB1 modulator is suitably fully miscible with the inhibitor. Without being bound by theory, this can be defined in the Bragg-Williams theory by the parameter χ being lower than 2.5, in particular lower than 2.

The χ parameter may be derived from the well known Bragg-Williams or the Regular Solution theories (see e.g. Jönsson, B. Lindman, K. Holmberg, B. Kronberg, “Surfactants and Polymers in Solution”, John Wiley & Sons, 1998 and Neau et al, Pharmaceutical Research, 14, 601 1997). In an ideal mixture χ is 0, and according to the Bragg-Williams theory a two-component mixture will not phase separate provided χ<².

As disclosed in WO 03/013272, when χ is equal or less than 2.5, concentrated particle dispersions that exhibit little or no Ostwald ripening, can be prepared. Those systems in which χ is larger than about 2.5 are thought to be prone to phase separation and are less stable against Ostwald ripening. Suitably the χ value of the (CB1 modulator)-inhibitor mixture is 2 or less, for example from 0 to 2, preferably 0.1 to 2, such as 0.2 to 1.8. However, the method of the present invention will not be bound by this theory.

Many small molecule organic substances (Mw<1000) are available in a crystalline form or can be prepared in crystalline form using conventional techniques (for example by recrystallisation from a suitable solvent system). In such cases the χ parameter of the CB1 modulator and inhibitor mixture is easily determined from Equation I:

$$χ = \frac{- Δ {S}_{m} ln [ {T}_{m} / T ] / R - ln {x}_{1}^{s}}{{( 1 - {x}_{1}^{s} )}^{2}} \qquad Equation I$$

wherein:
ΔSₘ is the entropy of melting of the crystalline substantially water-insoluble CB1 modulator (measured using a conventional technique such as DSC measurement);
Tₘ is the melting point (K) of the crystalline substantially water-insoluble CB1 modulator (measured using a conventional technique such as DSC measurement);
T is the temperature at the solubility experiment
R is the gas constant; and
x^{s}₁ is the mole fraction solubility of the crystalline substantially water-insoluble CB1 modulator in the inhibitor (measured using conventional techniques for determining solubility for example as hereinbefore described). In the above equation Tₘ and ΔSₘ refer to the melting point of the crystalline form of the material. In those cases where the CB1 modulator may exist in the form of different polymorphs, Tₘ and ΔSₘ are determined for the polymorphic form of the CB1 modulator that is used in the solubility experiment. As will be understood, the measurement of ΔSₘ, Tₘ and x^{s}₁ are performed on the crystalline substantially water-insoluble CB1 modulator prior to formation of the dispersion according to the invention and thereby enables a preferred inhibitor for the substantially water-insoluble material to be selected by performing simple measurements on the bulk crystalline material.

The mole fraction solubility of the crystalline substantially water-insoluble CB1 modulator in the inhibitor (x^{s}₁) is simply the number of moles of CB1 modulator per mole of inhibitor present in a saturated solution of the CB1 modulator in the inhibitor. As will be realized the equation above is derived for a two-component system of a CB1 modulator and an inhibitor. In those systems where the inhibitor contains more than one compound (for example in the case of a medium chain triglyceride comprising a mixture of triglycerides such as Miglyol 812N, or where a mixture of inhibitors is used) it is sufficient to calculate x^{s}₁ in terms of the “apparent molarity” of the mixture of inhibitors.

The apparent molarity of such a mixture is calculated for a mixture of inhibitor components to be:

Apparent molarity=Mass of 1 litre of inhibitor mixture*[(a/Mwa)+(b/Mwb)+ . . . (n/Mwn)]

wherein:
a, b . . . n are the weight fraction of each component in the inhibitor mixture (for example for component a this is % w/w component a/100); and
Mwa . . . . Mwn is the molecular weight of each component a . . . n in the mixture.
x^{s}₁ is then calculated as:

x^{s}₁=Molar solubility of the crystalline CB1 modulator in the inhibitor mixture (mol/l) Apparent molarity of inhibitor mixture (mol/l)

When the inhibitor is a solid at the temperature that the dispersion is prepared, the mole fraction solubility, x^{s}₁, can be estimated by measuring the mole fraction solubility at a series of temperatures above the melting point of the inhibitor and extrapolating the solubility back to the desired temperature. However, as hereinbefore mentioned, it is preferred that the inhibitor is a liquid at the temperature that the dispersion is prepared. This is advantageous because, amongst other things, the use of a liquid inhibitor enables the value of x^{s}₁ to be measured directly.

In certain cases, it may not be possible to obtain the substantially water-insoluble CB1 modulator in a crystalline form, particularly in the case of large organic molecules which may be amorphous. In such cases, preferred inhibitors are those which are sufficiently miscible with the substantially water-insoluble CB1 modulator to form a substantially single phase mixture (according to the theory above, χ<2.5, in particular χ<2) when mixed in the required CB1 modulator:inhibitor ratio. Miscibility of the inhibitor in the substantially water-insoluble CB1 modulator may be determined using routine experimentation. For example the CB1 modulator and inhibitor may be dissolved in a suitable organic solvent followed by removal of the solvent to leave a mixture of the CB1 modulator and inhibitor. The resulting mixture may then be characterised using a routine technique such as DSC characterisation to determine whether or not the mixture is a single-phase system. This empirical method enables preferred inhibitors for a particular CB1 modulator to be selected and will provide substantially single-phase particles in the dispersion prepared according to the present invention.

### The Co-Inhibitor

In a further embodiment of the process according to the invention a suitable co-inhibitor is present in the emulsion. In those cases, the inhibitor mixture comprising at least one inhibitor and at least one co-inhibitor is treated as a pseudo one-component mixture. The presence of the co-inhibitor increases the miscibility of the CB1 modulator and the inhibitor mixture, thereby reducing the χ value and further reducing or preventing Ostwald ripening. The co-inhibitor is suitably more soluble in water than the inhibitor. Suitable inhibitor mixtures include an inhibitor as hereinbefore is defined, preferably an inhibitor selected from classes (i) to (vi) listed hereinbefore. Examples of co-inhibitors are long-chain aliphatic alcohols, such as aliphatic alcohols containing 6 or more carbons, in particular from 6 to 14 carbon atoms, e.g. 1-hexanol and 1-decanol. In a preferred embodiment when the inhibitor is a medium chain triglyceride containing acyl groups with 8 to 12 carbon atoms (or a mixture of such triglycerides such as Miglyol 812N), a preferred co-inhibitor is a long chain aliphatic alcohol containing 6 or more carbon atoms (preferably from 6 to 14 carbon atoms) for example 1-hexanol or more preferably 1-decanol. Other suitable co-inhibitors include hydrophobic polymers, for example polypropylene glycol 2000, and hydrophobic block copolymers, for example the tri-block copolymer Pluronic L121. The weight ratio of inhibitor:co-inhibitor is selected to give the desired χ value of the mixture of the CB1 modulator and the inhibitor (mixture) and may be varied over wide limits, for example from 10:1 to 1:10 (w/w), for example 1:2 (w/w) and approximately 1:1 (w/w). Preferred values for χ are as hereinbefore defined.

In one embodiment of the present invention a stable dispersion of particles of a substantially water-insoluble CB1 modulator in an aqueous medium is provided. The dispersions prepared according to this embodiment exhibit little or no growth in particle size during storage resulting from Ostwald ripening.

In one embodiment it is preferred that the miscibility of the substantially water-insoluble CB1 modulator and the inhibitor mixture (comprising at least one inhibitor and at least one co-inhibitor) are sufficient to give substantially single phase particles in the dispersion, more preferably the mixture of said inhibitor mixture and CB1 modulator has a χ value of <2.5, more preferably 2 or less, for example from 0 to 2 wherein the χ value is as hereinbefore defined.

In one embodiment the inhibitor is preferably a medium chain tri-glyceride (MCT) containing acyl groups with 8 to 12 carbon atoms, more preferably 8 to 10 carbon atoms, or a mixture thereof, for example Miglyol 812N. The miscibility of the inhibitor with the CB1 modulator may be increased by using a co-inhibitor as hereinbefore described. For example, a suitable inhibitor/co-inhibitor in this embodiment comprises a medium chain tri-glyceride (MCT) as defined above and a long chain aliphatic alcohol having 6 to 12, more preferably 8 to 12, for example 10, carbon atoms, or a mixture comprising two or more such inhibitors, for example 1-hexanol or, more preferably, 1-decanol. A preferred mixture of inhibitor/co-inhibitor for use in this embodiment is a mixture of Miglyol 81 2N and 1-decanol.

If required the particles present in the dispersion prepared according to the present invention may be isolated from the aqueous medium. The particles may be separated using conventional techniques, for example by centrifuging, reverse osmosis, membrane filtration, lyophilisation or spray drying. Isolation of the particles is useful because it allows the particles to be washed and re-suspended in a sterile aqueous medium to give a suspension suitable for administration to a warm blooded mammal, especially a human, for example by oral or parenteral e.g. intravenous, administration.

In one embodiment an agent may be added to the suspension prior to isolation of the particles to prevent agglomeration of the solid particles during isolation, for example freezing, spray-drying, spray-granulation or lyophilisation and also during thawing. Suitable agents include for example a sugar, such as mannitol, trehalose or sucrose.

Isolation of the particles from the suspension is also useful when it is desirable to store the particles as a powder. The powder may then be re-suspended in an aqueous medium prior to use. The isolated particles of the CB1 modulator may then be stored as a powder in, for example, a vial and subsequently be re-suspended in a suitable liquid medium for administration to a patient as described above.

Alternatively the isolated particles may be used to prepare solid formulations, for example by blending the particles with suitable excipients/carriers and granulating or compressing the resulting mixture to form a tablet or granules suitable for oral administration. Alternatively the particles may be suspended, dispersed or encapsulated in a suitable matrix system, for example a biocompatible polymeric matrix, for example a hydroxypropyl methylcellulose (HPMC) or polylactide-co-glycloide polymer to give a controlled or sustained release formulation.

In another embodiment of the present invention the process may be performed at such high temperatures, that a sterile dispersion is provided directly, and which dispersion can be administered to a warm blooded mammal as described above without the need for additional purification or sterilisation steps.

According to a further aspect of the present invention a stable aqueous dispersion is provided comprising a continuous aqueous phase in which particles are dispersed. These dispersed particles comprise an inhibitor and a substantially water-insoluble CB1 modulator, and the said dispersion is obtainable by the process according to the present invention; and wherein:

(i) the inhibitor is a compound that is substantially insoluble in water;
(ii) the inhibitor is less soluble in water than the substantially water-insoluble CB1 modulator; and
the inhibitor is completely miscible with the amorphous phase of the substantially water-insoluble CB1 modulator.

The dispersion according to this aspect of the present invention exhibit little or no particle growth upon storage, mediated by Ostwald ripening (i.e. the dispersion is a stable dispersion as defined above), and reduced crystallization rate of the amorphous sub-micron particle.

The particles preferably have a mean diameter of less than 1 μm and more preferably less than 500 nm. It is especially preferred that the particles in the dispersion have a mean particle size of from 10 to 500 nm, more especially from 50 to 300 nm and still more especially from 100 to 200 nm.

The particles may contain a single substantially water-insoluble CB1 modulator, or two or more of such substances. The particles may contain a single inhibitor or a combination of an inhibitor and one or more co-inhibitors as hereinbefore described.

### Medical Use

The dispersions according to the present invention may be administered to a warm-blooded mammal (especially a human), for example by oral or parenteral (e.g. intravenous) administration. In an alternative embodiment the dispersion may be used as a granulation liquid in a wet granulation process to prepare granules comprising the substantially water-insoluble pharmacologically active material and one or more excipients, optionally after first concentrating the dispersion by removal of excess aqueous medium. The resulting granules may then be used directly, for example by filling into capsules to provide a unit dosage containing the granules. Alternatively the granules may be optionally mixed with further excipients, disintegrants, binders, lubricants etc. and compressed into a tablet suitable for oral administration. If required the tablet may be coated to provide control over the release properties of the tablet or to protect it against degradation, for example through exposure to light and or moisture. Wet granulation techniques and excipients suitable for use in tablet formulations are well known in the art.

According to a further aspect of the present invention there is provided a solid particle comprising an inhibitor and a substantially water-insoluble CB1 modulator obtainable by the process according to the present invention, wherein the CB1 modulator and the inhibitor are as hereinbefore defined.

According to a further aspect of the present invention there is provided a solid particle comprising an inhibitor and a substantially water-insoluble CB1 modulator obtainable by the process according to the present invention, for use as a medicament, wherein the CB1 modulator and the inhibitor are as hereinbefore defined.

According to a further aspect of the present invention there is provided a pharmaceutical composition comprising a pharmaceutically acceptable carrier or diluent in association with a solid particle comprising an inhibitor and a substantially water-insoluble CB1 modulator obtainable by the process according to the present invention.

Suitable pharmaceutically acceptable carriers or diluents are well known excipients used in the preparation of pharmaceutical formulations, for example, fillers, binders, lubricants, disintegrants and/or release controlling/modifying excipients.

The invention is further illustrated by the following examples in which all parts are parts by weight unless stated otherwise.

### EXAMPLES

Pluronic L121 and PVP 17 PF were obtained from BASF, Docusate Sodium (AOT) from Cytec and PVPK 30 and Sucrose from Sigma. N,N-Dimethylacetamide, (DMA) was obtained from Scharlau, D-Mannitol from RiedeldeHaen and Miglyol 812N from AstraZeneca.

### Example 1

1-propanesulfonic acid, 3,3,3-trifluoro-, 4-[1-(2,4-dichlorophenyl)-3-[[(2-hydroxycyclohexyl)amino]carbonyl]-4-(hydroxymethyl)-1H-pyrazol-5-yl]phenyl ester (A) −3.25% (w/w) A (drug/Miglyol/L121 3:1:2 (w/www)), 0.19% (w/w) AOT

An oil-in-water emulsion containing 20% (w/w) Miglyol 812N/Pluronic L121 (1:2 w/w) and 0.57% (w/w) docusate sodium (AOT) was prepared as follows. An oil-in-water emulsion containing 20% (w/w) Miglyol 812N and 1.7% (w/w) docusate sodium (AOT) was prepared using a Polytron homogenizer followed by high-pressure homogenization (Rannie). To this emulsion the co-inhibitor Pluronic L121 and water was added and mixed by stirring at approximately 8° C. for 12 h, interrupted by 3×5 minutes sonication, giving a final emulsion containing 6.7% (w/w) Miglyol 812N, 13.3% (w/w) PluronicL121 and 0.57% (w/w) AOT. The mean emulsion droplet size was measured using dynamic light scattering (Brookhaven FOQELS) to 145 nm.

A 6.5% (w/w) suspension of crystalline 1-propanesulfonic acid, 3,3,3-trifluoro-, 4-[1-(2,4-dichlorophenyl)-3-[[(2-hydroxycyclohexyl)amino]carbonyl]-4-(hydroxymethyl)-1H-pyrazol-5-yl]phenyl ester in water containing 0.19% (w/w) AOT was prepared by sonication and stirring, having a volume-averaged particle size of 5.4 μm, as measured by laser diffraction (Malvern Mastersizer 2000). 0.57 mL of the emulsion was mixed with 1.75 mL of the suspension and 1.18 ml of water and heated in high-pressure vials 170° C. for 15 minutes. The mixture was then cooled down to room temperature and the mean particle size measured with dynamic light scattering (Brookhaven FOQELS) to 205 nm.

### Example 2

1-propanesulfonic acid, 3,3,3-trifluoro-, 4-[3-[(cyclohexylamino)carbonyl]-1-(2,4-dichlorophenyl)-4-(hydroxymethyl)-1H-pyrazol-5-yl]phenyl ester (B) −3.25% (w/w) B (drug/Miglyol 1:1 (w/w)), 0.19% (w/w) AOT, 0.25% (w/w) PVPK30

An oil-in-water emulsion containing 20% (w/w) Miglyol 812N and 0.57% (w/w) docusate sodium (AOT) was prepared using a Polytron homogenizer followed by high-pressure homogenization (Rannie). The mean emulsion droplet size was measured using dynamic light scattering (Brookhaven FOQELS) to 155 nm.

A 6.5% (w/w) suspension of crystalline 1-propanesulfonic acid, 3,3,3-trifluoro-, 4-[3-[(cyclohexylamino)carbonyl]-1-(2,4-dichlorophenyl)-4-(hydroxymethyl)-1H-pyrazol-5-yl]phenyl ester in water containing 0.19% (w/w) AOT and 0.50% (w/w) PVPK30 was prepared by sonication and stirring, having a volume-averaged particle size of 10.5 μm, as measured by laser diffraction. 0.57 mL of the emulsion was mixed with 1.75 mL of the suspension and 1.18 ml of water and heated in high-pressure vials 170° C. for 15 minutes. The mixture was then cooled down to room temperature and the mean particle size measured with dynamic light scattering (Brookhaven FOQELS) to 250 nm.

### Example 3

1-propanesulfonic acid, 3,3,3-trifluoro-, 4-[1-(2,4-dichlorophenyl)-4,5,6,7-tetrahydro-3-methyl-4-oxo-5-(1-piperidinyl)-1H-pyrrolo[3,2-c]pyridin-2-yl]phenyl ester (C) −3% (w/w) C (drug/Miglyol 1:1 (w/w)), 0.17% (w/w) AOT

An oil-in-water emulsion containing 20% (w/w) Miglyol 812N and 0.57% (w/w) docusate sodium (AOT) was prepared using a Polytron homogenizer followed by high-pressure homogenization (Rannie). The mean emulsion droplet size was measured using dynamic light scattering (Brookhaven FOQELS) to 160 nm n.

A 6% (w/w) suspension of crystalline 1-propanesulfonic acid, 3,3,3-trifluoro-, 4-[1-(2,4-dichlorophenyl)-4,5,6,7-tetrahydro-3-methyl-4-oxo-5-(1-piperidinyl)-1H-pyrrolo[3,2-c]pyridin-2-yl]phenyl ester in water containing 0.17% (w/w) AOT and 0.50% (w/w) PVP K30 was prepared by sonication and stirring, having a volume-averaged particle size of 5.7 μm, as measured by laser diffraction. The emulsion was diluted to 6% (w/w) with water. 0.5 mL of the emulsion was mixed with 0.5 mL of the suspension and heated in high-pressure vials 160° C. for 10 minutes. The mixture was then cooled down to room temperature and the mean particle size measured with dynamic light scattering (Brookhaven FOQELS) to 220 nm.

### Example 4

### 1-propanesulfonic acid, 3,3,3-trifluoro-, 4-[1-(2,4-dichlorophenyl)-4-methyl-3-[(1-piperidinylamino)carbonyl]-1H-pyrazol-5-yl]phenyl ester (D) −3% (w/w) D (drug/Miglyol 1:1 (w/w)), 0.17% (w/w) AOT

An oil-in-water emulsion containing 20% (w/w) Miglyol 812N and 0.57% (w/w) docusate sodium (AOT) was prepared using a Polytron homogenizer followed by high-pressure homogenization (Rannie). The mean emulsion droplet size was measured using dynamic light scattering (Brookhaven FOQELS) to 160 nm.

A 6.0% (w/w) suspension of crystalline 1-propanesulfonic acid, 3,3,3-trifluoro-, 4-[1-(2,4-dichlorophenyl)-4-methyl-3-[(1-piperidinylamino)carbonyl]-1H-pyrazol-5-yl]phenyl ester in water containing 0.17% (w/w) AOT was prepared by sonication and stirring, having a volume-averaged particle size of 3.5 μm, as measured by laser diffraction. 0.15 mL of the emulsion was mixed with 0.50 mL of the suspension and 0.35 ml of water and heated in high-pressure vials 170° C. for 10 minutes. The mixture was then cooled down to room temperature and the mean particle size measured with dynamic light scattering (Brookhaven FOQELS) to 230 nm.

### Example 5

1-propanesulfonic acid, 3,3,3-trifluoro-, 4-[1-(2,4-dichlorophenyl)-4-methyl-3-[(1-piperidinylamino)carbonyl]-1H-pyrazol-5-yl]phenyl ester −3% (w/w) D (drug/Miglyol 1:1 (w/w)), 0.17% (w/w) AOT, 0.25% (w/w) PVP K30

An oil-in-water emulsion containing 20% (w/w) Miglyol 812N and 0.57% (w/w) docusate sodium (AOT) was prepared using a Polytron homogenizer followed by high-pressure homogenization (Rannie). The mean emulsion droplet size was measured using dynamic light scattering (Brookhaven FOQELS) to 160 nm.

A 6.0% (w/w) suspension of crystalline 1-propanesulfonic acid, 3,3,3-trifluoro-, 4-[1-(2,4-dichlorophenyl)-4-methyl-3-[(1-piperidinylamino)carbonyl]-1H-pyrazol-5-yl]phenyl ester in water containing 0.17% (w/w) AOT and 0.50% (w/w) PVP K30 was prepared by sonication and stirring, having a volume-averaged particle size of 3.9 μm, as measured by laser diffraction. 0.15 mL of the emulsion was mixed with 0.50 mL of the suspension and 0.35 ml of water and heated in high-pressure vials 170° C. for 10 minutes. The mixture was then cooled down to room temperature and the mean particle size measured with dynamic light scattering (Brookhaven FOQELS) to 205 nm.

### Example 6

### 1-propanesulfonic acid, 3,3,3-trifluoro-, 4-[1-(2,4-dichlorophenyl)-4-methyl-3-[(1-piperidinylamino)carbonyl]-1H-pyrazol-5-yl]phenyl ester (D) −3% (w/w) D (drug/Miglyol/L121 3:1:2 (w/w/w)), 0.17% (w/w) AOT

An oil-in-water emulsion containing 20% (w/w) Miglyol 812N/Pluronic L121 (1:2 w/w) and 0.57% (w/w) docusate sodium (AOT) was prepared as follows; an oil-in-water emulsion containing 20% (w/w) Miglyol 812N and 1.7% (w/w) docusate sodium (AOT) was prepared using a Polytron homogenizer followed by high-pressure homogenization (Rannie). To this emulsion the co-inhibitor Pluronic L121 and water was added and mixed by stirring at approximately 8° C. for 12 h, interrupted by 3×5 minutes sonication, giving a final emulsion containing 6.7% (w/w) Miglyol 812N, 13.3% (w/w) PluronicL121 and 0.57% (w/w) AOT. The mean emulsion droplet size was measured using dynamic light scattering (Brookhaven FOQELS) to 145 nm.

A 6.0% (w/w) suspension of crystalline 1-propanesulfonic acid, 3,3,3-trifluoro-, 4-[1-(2,4-dichlorophenyl)-4-methyl-3-[(1-piperidinylamino)carbonyl]-1H-pyrazol-5-yl]phenyl ester in water containing 0.17% (w/w) AOT was prepared by sonication and stirring, having a volume-averaged particle size of 5.1 μm, as measured by laser diffraction. 0.15 mL of the emulsion was mixed with 0.5 mL of the suspension and 0.35 ml of water and heated in high-pressure vials 170° C. for 10 minutes. The mixture was then cooled down to room temperature and the mean particle size measured with dynamic light scattering (Brookhaven FOQELS) to 250 nm.

### Example 7

1-propanesulfonic acid, 3,3,3-trifluoro-, 4-[1-(2,4-dichlorophenyl)-4-methyl-3-[(1-piperidinylamino)carbonyl]-1H-pyrazol-5-yl]phenyl ester (D) 3.0% (w/w) D (drug/Miglyol/L121 3:1:2 (w/w/w)), 0.17% (w/w) AOT, 0.13% (w/w) PVP K17

An oil-in-water emulsion containing 20% (w/w) Miglyol 812N/Pluronic L121 (1:2 w/w) and 0.57% (w/w) docusate sodium (AOT) was prepared as follows; an oil-in-water emulsion containing 20% (w/w) Miglyol 812N and 1.7% (w/w) docusate sodium (AOT) was prepared using a Polytron homogenizer followed by high-pressure homogenization (Rannie). To this emulsion the co-inhibitor Pluronic L121 and water was added and mixed by stirring at approximately 8° C. for 12 h, interrupted by 3×5 minutes sonication, giving a final emulsion containing 6.7% (w/w) Miglyol 812N, 13.3% (w/w) PluronicL121 and 0.57% (w/w) AOT. The mean emulsion droplet size was measured using dynamic light scattering (Brookhaven FOQELS) to 145 nm.

A 6.0% (wiw) suspension of crystalline 1-propanesulfonic acid, 3,3,3-trifluoro-, 4-[1-(2,4-dichlorophenyl)-4-methyl-3-[(1-piperidinylamino)carbonyl]-1H-pyrazol-5-yl]phenyl ester in water containing 0.17% (w/w) AOT and 0.25% (w/w) PVPK17 was prepared by sonication and stirring, having a volume-averaged particle size of 4.4 μm, as measured by laser diffraction. 0.15 mL of the emulsion was mixed with 0.5 mL of the suspension and 0.35 ml of water and heated in high-pressure vials 170° C. for 10 minutes. The mixture was then cooled down to room temperature and the mean particle size measured with dynamic light scattering (Brookhaven FOQELS) to 220 nm.

### Example 8

### 1-propanesulfonic acid, 4-[1-(2,4-dichlorophenyl)-4-methyl-3-[(1-piperidinylamino)carbonyl]-1H-pyrazol-5-yl]phenyl ester; (E) 10% (w/w) (E) (drug/MiglyoL/L121 3:1:2 (w/w/w), 0.56% (w/w) AOT

An oil-in-water emulsion containing 20% Miglyol/L121 1:2 (w/w) and 0.56% (w/w) AOT was prepared from a 1:2 (w/w) mixture of Miglyol 812N and L121 and a 0.7% (w/w) AOT solution by vigorous vortex mixing followed by repeated sonication and cooling to approximately 4° C. The mean droplet size was 140 nm, as measured with dynamic light scattering (Brookhaven FOQELS). A 20% (w/w) suspension of crystalline 1-propanesulfonic acid, 4-[1-(2,4-dichlorophenyl)-4-methyl-3-[(1-piperidinylamino)carbonyl]-1H-pyrazol-5-yl]phenyl ester in water containing 0.56% (w/w) AOT and 0.2% (w/w) PVP 12 PF was prepared by sonication and stirring. 0.45 mL of the emulsion was mixed with 0.45 mL of the suspension and heated in a high-pressure vial at 200° C. for 10 minutes. After heating, only a small fraction of phase-separated material was found and after cooling to room temperature the mean particle size was measured with dynamic light scattering (Brookhaven FOQELS) to 170 nm.

### Example 9

### 3,3,3-Trifluoropropane-1-sulfonic acid, 4-[2-(2,4-dichlorophenyl)-5-methyl-4-(piperidin-1-ylcarbamoyl)imidazol-1-yl]phenyl ester (F)

This example is prepared in a similar manner to Example 8.

10% (w/w) F (drug/Miglyo 1121 3:1:2 (w/w/w), 0.57% (w/w) AOT, 0.1% (w/W) PVP K12.

An oil-in-water emulsion containing 20% (w/w) Miglyol 812N and 1.7% (w/w) AOT was prepared using a Polytron homogenizer followed by high-pressure homogenization (Rannie). To this emulsion the co-inhibitor Pluronic L121 and water was added and mixed by stirring at approximately 8° C. for 12 h, interrupted by 3×5 minutes sonication, giving a final emulsion containing 6.7% (w/w) Miglyol 812N, 13.3% (w/w) Pluronic L121 and 0.57% (w/w) AOT. The mean droplet size was 155 nm, as measured with dynamic light scattering (Brookhaven FOQELS).

A 20% (w/w) suspension of crystalline 3,3,3-Trifluoropropane-1-sulfonic acid, 4-[2-(2,4-dichlorophenyl)-5-methyl-4-(piperidin-1-ylcarbamoyl)imidazol-1-yl]phenyl ester in water containing 0.56% (w/w) AOT and 0.2% (w/w) PVP K12 was prepared by sonication and stirring, having a volume-averaged particle size of 6.0 μm, as measured by laser diffraction. 0.5 mL of the emulsion was mixed with 0.5 mL of the suspension and heated in a high-pressure vial at 200° C. for 10 minutes. After heating, only a small fraction of phase separated material was found and after cooling to room temperature the mean particle size was measured with dynamic light scattering (Brookhaven FOQELS) to 290 nm.

### Example 10

3,3,3-Trifluoropropane-1-sulfonic acid 4-[1-(2-chloro-4-fluorophenyl)-3-methyl-4-oxo-5-piperidin-1-yl-4,5,6,7-tetrahydro-1H-pyrrolo[3,2-c]pyridin-2-yl]phenyl ester (G) 10% (w/w) G (drug/Miglyol/L121 3:1:2 (w/w/w), 0.57% (w/w) AOT, 0.1% (w/w) PVP K12

An oil-in-water emulsion containing 20% (w/w) Miglyol 812N and 1.7% (w/w) AOT was prepared using a Polytron homogenizer followed by high-pressure homogenization (Rannie). To this emulsion the co-inhibitor Pluronic L121 and water was added and mixed by stirring at approximately 8° C. for 12 h, interrupted by 3×5 minutes sonication, giving a final emulsion containing 6.7% (w/w) Miglyol 812N, 13.3% (w/w) Pluronic L121 and 0.57°% (w/w) AOT. The mean droplet size was 155 mn, as measured with dynamic light scattering (Brookhaven FOQELS).

A 20% (w/w) suspension of crystalline 3,3,3-Trifluoropropane-1-sulfonic acid 4-[1-(2-chloro-4-fluorophenyl)-3-methyl-4-oxo-5-piperidin-1-yl-4,5,6,7-tetrahydro-1H-pyrrolo[3,2-c]pyridin-2-yl]phenyl ester in water containing 0.56% (w/w) AOT and 0.2% (w/w) PVP K12 was prepared by sonication and stirring, having a volume-averaged particle size of 5.1 μm, as measured by laser diffraction. 0.5 mL of the emulsion was mixed with 0.5 mL of the suspension and heated in a high-pressure vial at 190° C. for 10 minutes. After heating, only a small fraction of phase separated material was found and after cooling to room temperature the mean particle size was measured with dynamic light scattering (Brookhaven FOQELS) to 160 nm.

### Preparation of CB1 Compounds

1-propanesulfonic acid, 3,3,3-trifluoro-, 4-[1-(2,4-dichlorophenyl)-3-[[(2-hydroxycyclohexyl)amino]carbonyl]-4-(hydroxymethyl)-1H-pyrazol-5-yl]phenyl ester was prepared as described in WO2006/067428.
1-propanesulfonic acid, 3,3,3-trifluoro-, 4-[3-[(cyclohexylamino)carbonyl]-1-(2,4-dichlorophenyl)-4-(hydroxymethyl)-1H-pyrazol-5-yl]phenyl ester was prepared as described in WO2006/067428.
3,3,3-Trifluoropropane-1-sulfonic acid 4-[2-(2,4-dichlorophenyl)-5-methyl-4-(piperidin-1-ylcarbamoyl)imidazol-1-yl]phenyl ester was prepared as described in WO2005/095354.
1-propanesulfonic acid, 3,3,3-trifluoro-, 4-[1-(2,4-dichlorophenyl)-4-methyl-3-[(1-piperidinylamino)carbonyl]-1H-pyrazol-5-yl]phenyl ester and 1-propanesulfonic acid, 4-[1-(2,4-dichlorophenyl)-4-methyl-3-[(1-piperidinylamino)carbonyl]-1H-pyrazol-5-yl]phenyl ester were prepared as described in WO2005/080328
1-propanesulfonic acid, 3,3,3-trifluoro-, 4-[1-(2,4-dichlorophenyl)-4,5,6,7-tetrahydro-3-methyl-4-oxo-5-(1-piperidinyl)-1H-pyrrolo[3,2-c]pyridin-2-yl]phenyl ester

### 1) Preparation of [1,1′]Bipiperidinyl-2,4-dione

### Step A 3-(Piperidin-1-ylamino)propionic acid methyl ester

To a solution of 1-aminopiperidine (100 g, 1.00 mol) in dry methanol at 0° C., methyl acrylate (99.0 ml, 1.10 mol) was added dropwise. The resulting mixture was stirred at room temperature overnight. After evaporation of the solvent, heptane was added to the residue, and the white solid (impurity) removed by filtration. The filtrate was concentrated to dryness to afford 80.0 g (43%) of the title compound as a yellow oil.

### Step B N-(2-Methoxycarbonylethyl)-N-piperidin-1-yl-maloamic acid ethyl ester

To a solution of 3-(piperidin-1-ylamino)propionic acid methyl ester (80.0 g, 0.43 mol) in dichloromethane was added triethylamine (71.0 ml, 0.50 mol) followed by slow addition of ethyl malonyl chloride (60.0 ml, 0.47 mol) at 0° C. The resulting slurry was stirred at room temperature for 4 hours. Water was added and the phases separated. The organic phase was dried (Na₂SO₄), filtered and concentrated. Flash chromatography (toluene:EtOAc 9:1-1:1) gave 81.0 g (63%) of the product as an oil used without further purification.

### Step C 2,4-Dioxo-[1,1′]bipiperidinyl-3-carboxylic acid ethyl ester

To a solution of N-(2-methoxycarbonylethyl)-N-piperidin-1-yl-maloamic acid ethyl ester (60.0 g, 0.20 mol) in a mixture of THF (1100 ml) and DMF (490 ml) was added cesium carbonate (195 g, 0.60 mol). The resulting mixture was boiled under reflux (80° C.) for 48 hours. The cooled reaction mixture was filtered and the filtrate evaporated. The combined filtered solid and the filtrate residue were purified by flash chromatography (CH₂Cl₂:MeOH 70:30) to give 15.0 g (28%) of the title compound as a pale yellow oil.

### Step D [1,1′]Bipiperidinyl-2,4-dione

The oil from step C was dissolved in 10% acetic acid (250 ml) and the solution boiled under reflux for one hour. The cooled reaction mixture was evaporated, and the residue purified by flash chromatography (CH₂Cl₂:acetone 9:1-1:1) to give 4.00 g (36%) of the title compound as a semi-solid.

2)

### Step 1 1-(2,4-Dichlorophenylamino)propan-2-one

A mixture of 2,4-dichloroaniline (20.2 g, 0.125 mol), iodoacetone (26.6 g, 0.145 mol) and potassium carbonate (18.1 g, 0.13 mol) in DMF (200 ml) was heated under nitrogen at 100° C. overnight. After cooling to room temperature (rt), water was added and the mixture extracted with ether (x3). The combined organic extracts were washed with water, dried (Na₂SO₄), filtered and concentrated. Flash chromatography (Heptane:EtOAc 90: 10-80:20) afforded 13.6 g (50%) of the title compound as a brown solid.

### Step 2 1-(2,4-Dichlorophenyl)-3-methyl-5-piperidin-1-yl-1,5,6,7-tetrahydro-pyrrolo[3,2-c]pyridine-4-one

To a solution of [1,1′]bipiperidinyl-2,4-dione (520 mg, 2.65 mmol) from 1) step D above in dry toluene (25 ml) at room temperature were added 1-(2,4-dichlorophenylamino)-propan-2-one (576 mg, 2.64 mmol) from Step 1 above followed by a catalytic amount ofp-TSA. The reaction mixture was boiled under reflux with a Dean-Stark trap, and 10 ml toluene was collected in the trap. One molar equivalent ofp-TSA (250 mg) was added and the reaction mixture was boiled under reflux for 5.5 hours. After cooling to room temperature, the reaction mixture was evaporated and purified by flash chromatography (heptane:EtOAc gradient) to give 250 mg (25%) of the title compound as a brown solid. A parallel experiment with 1.51 g 1-(2,4-dichlorophenylamino)propan-2-one afforded 0.68 g (26%) of the product.

### Step 3 2-Bromo-1-(2,4-Dichlorophenyl)-3-methyl-5-piperidin-1-yl-1,5,6,7-tetrahydro-pyrrolo[3,2-c]pyridine-4-one

To as solution of 1-(2,4-dichlorophenyl)-3-methyl-5-piperidin-1-yl-1,5,6,7-tetrahydropyrrolo[3,2-c]pyridine-4-one (0.93 g, 2.46 mmol) in DMF (25 ml) was added NBS (0.48, 2.71 mmol) at 0° C. The reaction mixture was stirred at this temperature for one hour and then water was added. The mixture was extracted with ether (x3). The combined ether extracts were dried (Na₂SO₄), filtered and concentrated to give 0.45 g (40%) of the title compound after flash chromatography (heptane:EtOAc gradient).

### Step 4 1-(2,4-Dichlorophenyl)-2-(4-hydroxyphenyl)-3-methyl-5-piperidin-1-yl-1,5,6,7-tetrahydro-pyrrolo[3,2-c]pyridine-4-one

2-Bromo-1-(2,4-Dichlorophenyl)-3-methyl-5-piperidin-1-yl-1,5,6,7-tetrahydropyrrolo[3,2-c]pyridine-4-one (450 mg, 0.98 mmol), 4-hydroxyphenylboronic acid (150 mg, 1.09 mmol) and tetrakis(triphenylphosphine)palladium(0) (150 mg) were dissolved in DME (20 ml) and 1 M Na₂CO₃ (5 ml)). The resulting solution was degassed and heated at 60° C. under nitrogen overnight. Water and EtOAc were added after cooling and the aqueous phase extracted with EtOAc (x3). The combined organic extracts were dried (Na₂SO₄), filtered and concentrated to give a crude product that was purified by flash chromatography (heptane:EtOAc gradient) to afford 0.40 g (87%) of the product as a pale yellow solid.

### Step 5 3,3,3-Trifluoropropane-1-sulfonic acid 4-[1-(2,4-dichlorophenyl)-3-methyl-4-oxo-5-piperidin-1-yl-4,5,6,7-tetrahydro-1H-pyrrolo[3,2-c]pyridine-2-yl]phenyl ester

To a solution of 1-(2,4-dichlorophenyl)-2-(4-hydroxyphenyl)-3-methyl-5-piperidin-1-yl-1,5,6,7-tetrahydropyrrolo[3,2-c]pyridine-4-one (0.40 g, 0.85 mmol) in dichloromethane (20 ml) at 0° C. was added triethylamine (0.14 ml, 1.02 mmol) followed by 3,3,3-trifluoropropanesulfonyl chloride (0.20 g, 1.02 mmol). The reaction mixture was subsequently stirred at room temperature for two hours. Concentration and purification by flash chromatography (heptane:EtOAc gradient) afforded 200 mg (37%) of the title compound as a colorless solid.

¹H NMR (CDCl₃): δ 7.51 (1H, m), 7.34-7.02 (6H, m), 3.75 (2H, m), 3.53-3.44 (3H, m), 3.40-3.00 (2H, broad s), 2.87-2.60 (5H, m), 2.41 (3H, s), 1.80-1.50 (6H, m), 1.40-1.20 (2H, m). MS: 630 (M+H). HPLC: 95%.

3,3,3-Trifluoropropane-1-sulfonic acid 4-[1-(2-chloro-4-fluorophenyl)-3-methyl-4-oxo-5-piperidin-1-yl-4,5,6,7-tetrahydro-1H-pyrrolo[3,2-c]pyridin-2-yl]phenyl ester

### Step 1 4-(2-Chloro-4-fluorophenylamino)-5,6,3′,4′,5′,6′-hexahydro-2′H-[1,1′]bipyridinyl-2-one

[1,1′]-Bipiperidinyl-2,4-dione (2.00 g, 10.19 mmol) was dissolved in toluene (8 ml) and 2-chloro-4-fluorophenylamine (1.78 g, 12.23 mmol) was added. More toluene (5 ml) was added. The reaction mixture was boiled under reflux at 110° C. for 17 h then allowed to cool. When the reaction mixture reached rt the product precipitated and it was collected by filtration to yield a beige solid (1.80 g, 55%).

¹H-NMR (400 MHz, CDCl₃) δ 7.41-7.32 (1H, m), 7.18-7.09 (1H, m), 7.00-6.90 (1H, m), 5.51 (1H, s), 5.11 (1H, s), 3.54 (2H, t), 3.38-2.67 (4H, br), 2.57 (2H, t), 1.74-1.50 (4H, m), 1.43-1.30 (2H, m).

### Step 2 3-{2-[4-(tert-Butyldimethylsilanyloxy)phenyl]-1-methyl-2-oxo-ethyl}-4-(2-chloro-4-fluorophenylamino)-5,6,3′,4′,5′,6′-hexahydro-2H-[1,1′]bipyridinyl-2-one

NaH (0.15 g, 6.25 mmol) was placed in a flask under nitrogen and dry THF (5 ml) was added. The mixture was cooled to 0° C. with an icebath and 4-(2-chloro-4-fluoro-phenylamino)-5,6,3′,4′,5′,6′-hexahydro-2′H-[1,1′]bipyridinyl-2-one (0.70 g, 2.16 mmol) suspended in dry THF (8 ml) was added dropwise. After 1 h 40 min tetrabutylammonium iodide (0.085 g, 0.23 mmol) was added followed by dropwise addition of 2-bromo-1-[4-(tert-butyldimethylsilanyloxy)phenyl]propan-1-one (1.122 g, 3.27 mmol) dissolved in dry THF (2 ml). The icebath was removed after the last addition. The reaction was continued at rt for 4 h whereafter the reaction was quenched by adding phosphate buffer pH 7.0. The THF was evaporated. DCM/water were added and the phases separated. DCM was evaporated from the dried organic layer to yield the crude product as an orange solid (crude 1.135 g).

### Step 3 2-[4-(tert-Butyldimethylsilanyloxy)phenyl]-1-(2-chloro-4-fluorophenyl)-3-methyl-5-piperidin-1-yl-1,5,6,7-tetrahydro-pyrrolo[3,2-c]pyridin-4-one

3-{2-[4-(tert-Butyldimethylsilanyloxy)phenyl]-1-methyl-2-oxo-ethyl}-4-(2-chloro-4-fluorophenylamino)-5,6,3′,4′,5′,6′-hexahydro-2′H-[1,1′]bipyridinyl-2-one (1.135 g, 1.94 mmol) was suspended in toluene (5 ml) and toluene-4-sulfonic acid (0.037 g, 0.19 mmol) was added. The reaction mixture was heated in a microwave oven at 100° C. for 30 min. Water/toluene were added to the reaction mixture and the phases separated. The organic phase was washed with water, dried (MgSO₄), filtered and evaporated to yield the crude product (crude 0.929 g).

### Step 4 1-(2-Chloro-4-fluoro-phenyl)-2-(4-hydroxy-phenyl)-3-methyl-5-piperidin-1-yl-1 5,6,7-tetrahydro-pyrrolo[3,2-c]pyridin-4-one

2-[4-(tert-Butyldimethylsilanyloxy)phenyl]-1-(2-chloro-4-fluorophenyl)-3-methyl-5-piperidin-1-yl-1,5,6,7-tetrahydropyrrolo[3,2-c]pyridin-4-one (0.929 g, 1.63 mmol) was suspended in THF (10 ml) and TBAF (1M in THF, 1.64 ml) was added. The reaction mixture was stirred at rt for 1 h whereafter the solvent was evaporated and ethyl acetate/water added. The phases were separated and the organic phase dried and evaporated. The crude product was recrystallised from ethyl acetate/toluene to yield the product as an orange solid (0.223 g, 23% over 3 steps).

### Step 5 3,3,3-Trifluoropropane-1-sulfonic acid 4-[1-(2-chloro-4-fluorophenyl)-3-methyl-4-oxo-5-piperidin-1-yl-4,5,6,7-tetrahydro-1H-pyrrolo[3,2-c]pyridin-2-yl]phenyl ester

1-(2-Chloro-4-fluorophenyl)-2-(4-hydroxyphenyl)-3-methyl-5-piperidin-1-yl-1,5,6,7-tetrahydropyrrolo[3,2-c]pyridin-4-one (0.223 g, 0.49 mmol) was co-concentrated with pyridine twice and put under nitrogen. Pyridine (2.5 ml) was added and the reaction mixture cooled to 0° C. with an ice-bath followed by addition of 3,3,3-trifluoropropane-1-sulfonyl chloride (0.153 g, 0.78 mmol). The reaction mixture was stirred at 0° C. for 3 h adding more 3,3,3-trifluoropropane-1-sulfonyl chloride (0.171 g, 0.87 mmol) after 1 h 10 min. The ice bath was removed and the reaction mixture evaporated. The crude product was purified by hplc to yield the product as a beige solid after freeze-drying (0.19 g, 63%).

HRMS Calcd for [C₂₈H₂₈ClF₄N₃O₄S+H]⁺:614.150. Found: 614.150.

¹H-NMR (400 MHz, CD₃OD) δ 7.33-7.25 (2H, m), 7.20-7.10 (4H, m), 7.08-7.01 (1H, m), 3.67-3.54 (4H, m), 3.10-2.67 (6H, m), 2.59 (2H, t), 2.22 (3H, s), 1.83-1.49 (4H, m), 1.49-1.19 (2H, m).

### In Vivo Testing

The dispersions prepared according to Example 1 and Example 2 were tested in in vivo rat study together with dispersions comprising crystalline particles (prepared by wet milling) of compound A and B, respectively. The dispersions were orally administered at 100 mmol/kg. The mean particle size of crystalline compound A was 200 nm, and the mean particle size of crystalline compound B was 210 nm.

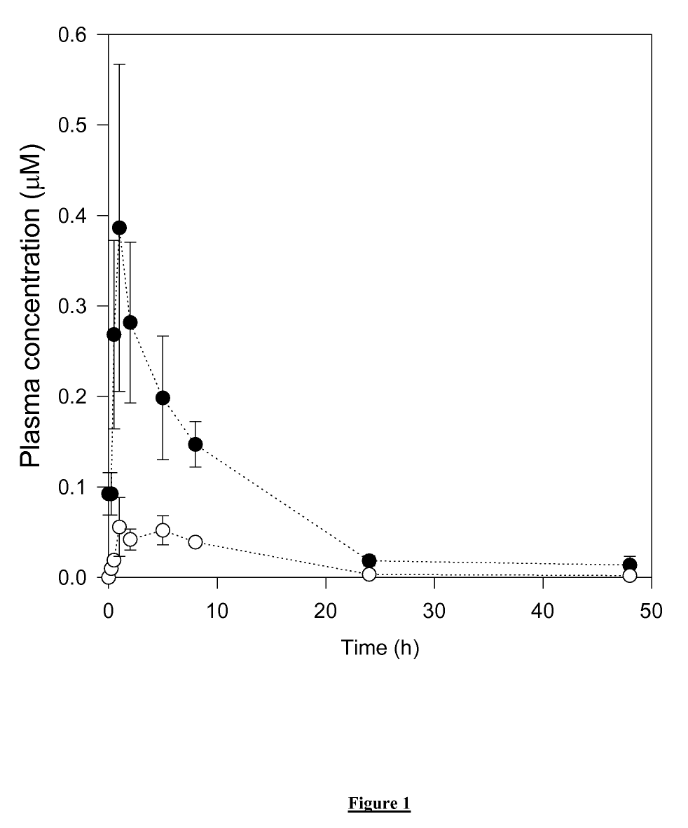

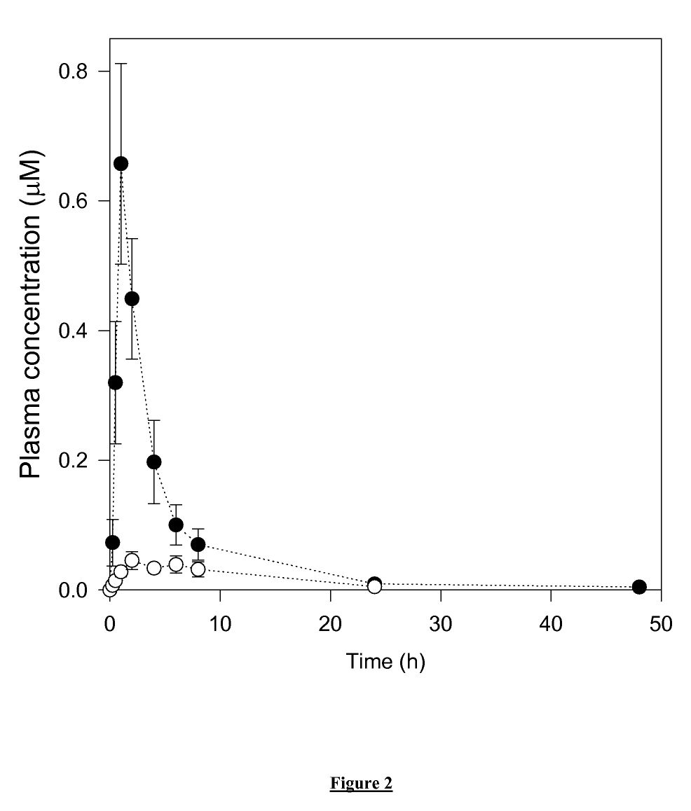

The results are shown in FIGS. 1 and 2, respectively. The Figures show the plasma concentration (pM) in the rats as a function of time (h). The filled circles illustrate the results obtained upon administration of the dispersions according to the invention, i.e. dispersions comprising amorphous particles. The open circles illustrate the results obtained upon administration of the dispersions comprising crystalline particles. The AUC (=area under curve) ratio and the maximum plasma concentration (c^{max}) ratios, respectively, are provided in Table 1.

TABLE 1

| | AUCₐₘₒᵣₚₕ/AUC_{crystal} | c^{max}ₐₘₒᵣₚₕ/c^{max}_{crystal} |
|---|---|---|
| Compound A | 4.8 | 6.9 |
| Compound B | 4.4 | 10 |

### Abbreviations

DCM dichloromethane
DME dimethoxyethane
DMF dimethylformamide
EtOAc ethyl acetate
NBS N-bromosuccinimide
MeOH methanol
p-TSA toluene-4-sulphonic acid
rt room temperature
TBAF tetrabutylammonium fluoride
TEA triethylamine
THF tetrahydrofuran
t triplet
s singlet
d doublet
q quartet
qvint quintet
m multiplet
br broad
bs broad singlet
dm doublet of multiplet
bt broad triplet
dd doublet of doublet

1. A process for the preparation of a stable dispersion of amorphous particles of a CB1 modulator of sub-micron size in an aqueous medium comprising the following steps:
1) combining
a) an emulsion comprising
an aqueous medium providing a continuous aqueous phase;
an inhibitor providing an oil phase and inhibiting particle growth due to flux of material between the particles dispersed in the aqueous medium;
a stabiliser preventing aggregation of emulsion droplets and optionally said particles;
with
b) a substantially water-insoluble CB1 modulator present in amorphous and/or crystalline state; wherein the ratio of substantially water-insoluble CB1 modulator to inhibitor is below 10:1 (w/w); and
c) optionally a second stabiliser preventing aggregation of emulsion droplets and/or said particles,
2) if any CB1 modulator in the crystalline state is present, increasing the temperature of the resulting mixture to the vicinity of the melting temperature of the crystalline CB1 modulator, and
3) allowing the CB1 modulator to migrate to said oil phase, and if the temperature was increased in step 2), decreasing the temperature, thereby providing the stable dispersion of amorphous particles.

2. A process according to claim 1 wherein the growth of the particles dispersed in said aqueous medium is less than 10% of the mean particle size over a period of 1 hour at ambient temperature after said preparation.

3. A process according to claim 1 wherein the substantially water-insoluble CB1 modulator is added in its crystalline state.

4. A process according to claim 1 wherein the substantially water-insoluble CB1 modulator is added in its amorphous state.

5. A process according to claim 1 wherein the substantially water-insoluble CB1 modulator is added as a suspension.

6. A process according to claim 5 wherein said second stabiliser is added to the suspension.

7. A process according to claim 1 wherein the stabiliser(s) is (are) selected from the group consisting of a polymeric dispersant, a surfactant and any mixture thereof.

8. A process according to claim 1 wherein the aqueous phase comprises a stabiliser in amount of 0.01 to 10% by weight.

9. A process according to claim 8 wherein the stabiliser is docusate sodium.

10. A process according to claim 8 wherein the stabiliser is a polyvinylpyrrolidone.

11. A process according to claim 1 wherein the aqueous medium consists of water.

12. A process according to claim 1 wherein step 2) is performed under high pressure.

13. A process according to claim 1 wherein the inhibitor is sufficiently miscible with the substantially water-insoluble CB1 modulator in amorphous state to form particles in the dispersion comprising a substantially single phase mixture of the CB1 modulator and the inhibitor.

14. A process according to claim 1 wherein a mixture of the inhibitor and the substantially water-insoluble CB1 modulator in amorphous state exhibits an interaction parameter χ, according to the Bragg-Williams theory, of less than 2.5.

15. A process according to claim 1 wherein a mixture of the inhibitor and the substantially water-insoluble CB1 modulator in amorphous state exhibits an interaction parameter X, according to the Bragg-Williams theory, of less than 2.

16. A process according to claim 1 wherein the inhibitor is less soluble in water than the substantially water-insoluble CB1 modulator.

17. A process according to claim 1 wherein the inhibitor has a water solubility at 25° C. of less than 0.1 mg/l.

18. A process according to claim 1 wherein the inhibitor is selected from the group consisting of mono-, di- or triglyceride of fatty acids, fatty acid mono- or di-ester of a C₂₋₁₀ diol, fatty acid esters of alkanols or cycloalkanols, waxes, long chain aliphatic alcohols and hydrogenated vegetable oils, or a combination of two or more inhibitors.

19. A process according to claim 18 wherein the inhibitor is a mixture of triglycerides obtainable by esterifying glycerol with a mixture of medium chain fatty acids.

20. A process according to claim 19 wherein the inhibitor is selected from medium chain triglycerides containing acyl groups with 8 to 12 carbon atoms.

21. A process according to claim 20 wherein the inhibitor is selected from the group consisting of Miglyol 810N, Miglyol 812N, Miglyol 818N, and any mixture thereof.

22. A process according to claim 21 wherein the inhibitor consists of Miglyol 812N.

23. A process according to claim 1 wherein the ratio of substantially water-insoluble CB1 modulator and inhibitor is 2:1 w/w by weight.

24. A process according to claim 1 wherein the ratio of substantially water-insoluble CB1 modulator and inhibitor is 1:1 w/w by weight.

25. A process according to claim 1 wherein the emulsion in step 1a) further comprises a co-inhibitor.

26. A process according to claim 25 wherein a mixture of the inhibitor and the co-inhibitor is sufficiently miscible with the substantially water-insoluble CB1 modulator in amorphous state to form particles in the dispersion comprising a substantially single phase mixture of the CB1 modulator, the inhibitor and the co-inhibitor.

27. A process according to claim 25 wherein a mixture of the inhibitor, the co-inhibitor and the substantially water-insoluble CB1 modulator in amorphous state exhibits an interaction parameter χ, according to the Bragg-Williams theory, of less than 2.5.

28. A process according to claim 25 wherein a mixture of the inhibitor, the co-inhibitor and the substantially water-insoluble CB1 modulator in amorphous state exhibits an interaction parameter χ, according to the Bragg-Williams theory, of less than 2.

29. A process according to claim 25 wherein the co-inhibitor is selected from the group consisting long chain aliphatic alcohols containing 6 or more carbon atoms, hydrophobic polymers and block copolymers, and any mixture thereof.

30. A process according to claim 25 wherein the inhibitor is a medium chain triglycerides containing acyl groups with 8 to 12 carbon atoms and the co-inhibitor is a long chain aliphatic alcohol containing 6 to 14 carbon atoms.

31. A process according to claim 25 wherein the co-inhibitor is selected from the group consisting of 1-hexanol, 1-decanol, and any mixture thereof.

32. A process according to claim 25 wherein the co-inhibitor is propylene glycol 2000.

33. A process according to claim 25 wherein the co-inhibitor is Pluronic L121.

34. A process according to claim 25 wherein the co-inhibitor is more soluble in water than the inhibitor.

35. A process according to claim 1 further comprising a step of isolating the amorphous particles in solid form from the dispersion.

36. A process according to claim 1 wherein the temperature in step 2) is increased to a temperature of +20° C. of the melting temperature of the crystalline CB1 modulator.

37. A process according to claim 1 wherein the CB1 modulator has a solubility in water at 25° C. of less than 0.5 mg/ml.

38. A process according to claim 1 wherein the CB1 modulator is a compound of formula (I) 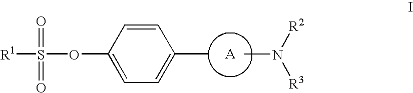
in which
R¹ represents a C₃₋₆alkyl group optionally substituted by one or more fluoro;
R² represents H and R³ represents cyclohexyl optionally substituted by hydroxy or R² and
R³ together with the nitrogen atom to which they are attached represent a piperidine ring which is optionally substituted by hydroxy; 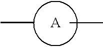
represents a group of formula a, b or c 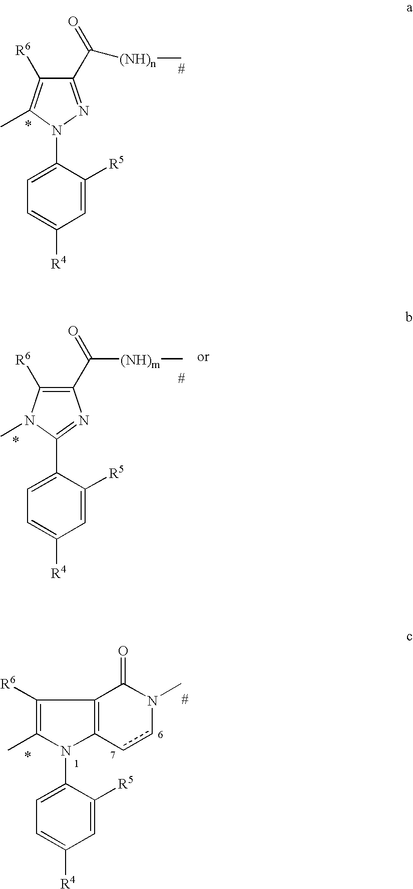
in which the bond marked * is attached to the phenyl ring carrying the sulphonyloxy group and the other bond marked # is attached to NR²R³; [symbol]
is an optional additional bond between positions 6 and 7 in formula c;
R⁴ and R⁵ independently represent H, bromo, chloro or fluoro; and
R⁶ represents methyl or hydroxymethyl;
n and m independently represent 0 or 1;
or a pharmaceutically acceptable salt thereof.

39. A process according to claim 38 wherein the CB1 modulator is a compound selected from:
1-propanesulfonic acid, 3,3,3-trifluoro-, 4-[1-(2,4-dichlorophenyl)-3-[[(2-hydroxycyclohexyl)amino]carbonyl]-4-(hydroxymethyl)-1H-pyrazol-5-yl]phenyl ester;
1-propanesulfonic acid, 3,3,3-trifluoro-, 4-[3-[(cyclohexylamino)carbonyl]-1-(2,4-dichlorophenyl)-4-(hydroxymethyl)-1H-pyrazol-5-yl]phenyl ester;
1-propanesulfonic acid, 3,3,3-trifluoro-, 4-[1-(2,4-dichlorophenyl)-4,5,6,7-tetrahydro-3-methyl-4-oxo-5-(1-piperidinyl)-1H-pyrrolo[3,2-c]pyridin-2-yl]phenyl ester;
1-propanesulfonic acid, 3,3,3-trifluoro-, 4-[1-(2,4-dichlorophenyl)-4-methyl-3-[(1-piperidinylamino)carbonyl]-1H-pyrazol-5-yl]phenyl ester;
1-propanesulfonic acid, 4-[1-(2,4-dichlorophenyl)-4-methyl-3-[(1-piperidinylamino)carbonyl]-1H-pyrazol-5-yl]phenyl ester;
3,3,3-trifluoropropane-1-sulfonic acid, 4-[2-(2,4-dichlorophenyl)-5-methyl-4-(piperidin-1-ylcarbamoyl)imidazol-1-yl]phenyl ester; or
3,3,3-trifluoropropane-1-sulfonic acid 4-[1-(2-chloro-4-fluorophenyl)-3-methyl-4-oxo-5-piperidin-1-yl-4,5,6,7-tetrahydro-1H-pyrrolo[3,2-c]pyridin-2-yl]phenyl ester or a pharmaceutically acceptable salt thereof.

40. A stable dispersion of amorphous submicron particles of a substantially water-insoluble CB1 modulator in an aqueous medium, obtainable by the process according to claim 1, and containing at least 1% by weight of the CB1 modulator.

41. A dispersion according to claim 40 containing from 1 to 30% by weight of the CB1 modulator.

42. The dispersion according to claim 40 for use as a medicament.

43. A pharmaceutical composition comprising the dispersion according to claim 40 in association with a pharmaceutically acceptable carrier or diluent.
